(19) United States
(12) Patent Application Publication
(10) Pub. No.: US 2026/0049990 A1
(43) Pub. Date: Feb. 19, 2026

(54) BIOMARKER FOR DIAGNOSING IMMUNE-RELATED DISEASES USING ASPARTATE METABOLITE

(71) Applicants: SUNGKWANG MEDICAL FOUNDATION, Seoul (KR); KONKUK UNIVERSITY INDUSTRIAL COOPERATION CORP, Seoul (KR)

(72) Inventors: Kyu Seok KIM, Seoul (KR); Choong Hwan LEE, Seoul (KR); Tae Nyoung CHUNG, Seoul (KR); Min Ji LEE, Yongin-si Gyeonggi-do (KR)

(73) Assignees: SUNGKWANG MEDICAL FOUNDATION, Seoul (KR); KONKUK UNIVERSITY INDUSTRIAL COOPERATION CORP, Seoul (KR)

(21) Appl. No.: 19/102,906

(22) PCT Filed: Aug. 11, 2023
(86) PCT No.: PCT/KR2023/011909

§ 371 Date: Feb. 26, 2025

(30) Foreign Application Priority Data
Aug. 11, 2022 (KR) 10-2022-0100520

(51) Int. Cl. G01N 33/68; G01N 30/72

(52) U.S. Cl. CPC G01N 33/68; G01N 30/7206; G01N 2560/00; G01N 2800/26

(57) ABSTRACT

The present disclosure relates to a biomarker for diagnosing immune-related diseases, a composition and kit for detecting the biomarker for diagnosing immune-related diseases, and a method of diagnosing immune-related disease by using the biomarker, composition and kit. According to the composition and kit for diagnosing immune-related diseases in an aspect and the method of diagnosing immune-related diseases by using the biomarker, composition and kit, the level of immune-related diseases in a subject can be easily diagnosed, and thus the health of the subject can be monitored and immune-related diseases can be prevented and diagnosed.

### TECHNICAL FIELD

The present disclosure relates to a biomarker for diagnosing immune-related diseases, a composition and kit for detecting the same for diagnosing immune-related diseases, and a method of diagnosing immune-related diseases by using the biomarker, composition and kit.

### BACKGROUND ART

Sepsis is an acute, severe disease in which a patient becomes infected with sepsis-causing pathogens, such as bacteria, viruses, fungi, and the like, suffering the patient from a high fever and eventually causing death. Here, there are two main causes of death. First, the so-called ‘cytokine storm’ causes the immune function to operate excessively, and such excessive autoimmune function destroys organs in one's body (hyperinflammation). Second, the reproduction of pathogens cannot be suppressed so that the pathogens spread in both the blood and organs, paralyzing the organs from functioning altogether (immune paralysis).

No fundamental medicine for the treatment of sepsis has been found as of yet. Sepsis is not easily cured by conventional anti-inflammatory treatments, and even the only FDA-approved drug, drotrecogin alfa (Xigris®, Engl. Ranieri, V. M. et al., 2012), has not been shown to have a clear therapeutic effect on sepsis in clinical trials, and thus research on it has been discontinued. Currently, sepsis is mainly treated with antibiotics or antifungal injections, and treatment drugs and duration are determined according to the type of microorganisms. In addition, depending on the patient's condition, hemodialysis or blood transfusions may also be performed. Sepsis can be cured with effective antibiotics and antifungal drugs, but in cases where a patient is infected with a drug-resistant microorganism, a patient is immunocompromised, and a patient is treated too late, treatment is difficult and the patient may die. In addition, severe sepsis and septic shock are, despite advances in variety of intensive care, including antibiotic therapy (Anderson, R. N. 2002; Andreu Ballester, J. C. et al., 2008; Angus, D. C. et al., 2001), the third leading cause of death in developed countries. Therefore, diagnosing and treating early sepsis symptoms before they progress to severe sepsis or septic shock may be critical to reducing such septic mortality rates. However, there are many difficulties in diagnosing early sepsis symptoms because there are no well-known markers for diagnosing sepsis.

Meanwhile, by confirming that levels of various metabolites change according to the progression of sepsis, the inventors of the present disclosure were able to complete the present disclosure as a biomarker composition for diagnosing sepsis or diseases involving immunosuppression.

### DISCLOSURE

### Technical Problem

One aspect is to provide a composition for diagnosing an immunosuppression level, the composition comprising an agent measuring a level of aspartic acid metabolite.

Another aspect is to provide a kit for diagnosing an immunosuppression level, the kit comprising an agent measuring a level of aspartic acid metabolite.

Another aspect is to provide a method of diagnosing an immunosuppression level in a subject, the method comprising measuring a level of aspartic acid metabolite in a biological sample separated from a subject.

Another aspect is to provide a composition for diagnosing an immunosuppression level, the composition comprising an agent measuring a level of at least one selected from the group consisting of glycine, aspartic acid, L-proline, glutamic acid, L-lysine, threonic acid, myo-inositol, and phosphorylethanolamine.

### Technical Solution

One aspect provides a composition for diagnosing an immunosuppression level, the composition comprising an agent measuring a level of aspartic acid metabolite.

The composition may be for diagnosing an immunosuppression level or immune-related diseases. Here, the diagnosing an immunosuppression level is diagnosing immune-related diseases or immunosuppression levels.

In an embodiment, the aspartic acid metabolite may be selected from the group consisting of glycine, aspartic acid, L-proline, glutamic acid, and L-lysine, and may include functional equivalents of the foregoing.

In an embodiment, the composition for diagnosing an immunosuppression level may include an agent measuring a level of at least one selected from the group consisting of threonic acid, myo-inositol, and phosphorylethanolamine.

In an embodiment, the immune-related diseases may include a disease involving immunosuppression or immune paralysis.

In an embodiment, the immune-related diseases may include sepsis.

In the present specification, the term “diagnosing” may refer to determining susceptibility of a subject to a particular disease or condition, determining whether a subject has a particular disease or condition, determining prognosis of a subject having a particular disease or condition, or monitoring the prognosis, and may be referred to as assessing or measuring an hyperimmunization or immunosuppression level.

In the present specification, the term “marker” or “biomarker” refers to a substance that can distinguish a subject with immune-related diseases and a subject without immune-related diseases for evaluation, and may include organic biomolecules, such as polypeptides, nucleic acids (e.g., mRNA, etc.), lipids, glycolipids, glycoproteins, sugars (monosaccharides, disaccharides, oligosaccharides, etc.), metabolites, and the like that are increased or reduced in a subject with immune-related diseases compared to a subject without immune-related disease. A marker for diagnosing immune-related diseases or an immunosuppression level of a subject provided by one aspect may be an aspartic acid metabolite whose expression level is reduced in a sample of a subject with immune-related diseases compared to a subject without immune-related diseases.

In the present specification, the term “metabolite” refers to a metabolite obtained from a sample of biological origin. The sample of biological origin from which the metabolite can be obtained may be preferably whole blood, plasma, serum, a platelet, a peripheral blood mononuclear cell, or a splenocyte, and more preferably a peripheral blood mononuclear cell or a splenocyte. The metabolite may include a substance produced by metabolism and metabolic processes, or a substance resulting from chemical metabolism by biological enzymes and molecules.

In the present specification, the term “aspartic acid (Asp)” refers to a type of amino acids that are involved in the metabolism of both the TCA cycle and the ornithine cycle. In the present specification, the term “measuring a level” refers to a process of determining an expression level of aspartic acid metabolites in a biological sample to diagnose an immunosuppression level. According to an embodiment of the present disclosure, a level of glycine, aspartic acid, L-proline, glutamic acid, L-lysine, threonic acid, myo-inositol, or phosphorylethanolamine metabolites may be determined.

The composition may further include a sample necessary for diagnosing an immunosuppression level.

Another aspect provides a kit for diagnosing immune-related diseases or an immunosuppression level, the kit comprising an agent measuring a level of aspartic acid metabolite.

In an embodiment, the aspartic acid metabolite may be selected from the group consisting of glycine, aspartic acid, L-proline, glutamic acid, and L-lysine, and may include functional equivalents of the foregoing.

In an embodiment, the kit for diagnosing an immunosuppression level may include an agent measuring a level of at least one selected from the group consisting of threonic acid, myo-inositol, and phosphorylethanolamine.

The aspartic acid metabolite and the level of metabolite may be the same as described above.

The kit may further include a sample necessary for diagnosing an immunosuppression level.

In an embodiment, the kit may be an integrated magneto-electrochemical sensor kit, but is not limited thereto. An integrated magneto-electrochemical sensor refers to a system that detects an electrical signal by enzymatic signal amplification using magnetic beads. Specifically, it may refer to a system in which a substance (e.g., an antibody) capable of detecting a target substance is attached to a magnetic bead to detect an electrical signal by enzymatic signal amplification generated by a chromogenic substance (e.g., horse radish peroxidase (HRP), alkaline phosphatase (ALP), α-D-galactosidase (α-Gal)) capable of detecting the target substance.

In an embodiment, the kit may include an agent measuring the level of aspartic acid metabolite, a device, and a computer with an embedded algorithm, and may relate to a kit that correlates the results of measuring the level of the marker with immunosuppression through the algorithm.

Another aspect provide a method of diagnosing immunosuppression levels in a subject, the method comprising measuring a level of an aspartic acid metabolite in a biological sample that is isolated from a subject.

In an embodiment, the aspartic acid metabolite may be selected from the group consisting of glycine, aspartic acid, L-proline, glutamic acid, and L-lysine, and may include functional equivalents of the foregoing.

In an embodiment, the diagnosing of immunosuppression levels comprises measuring levels of at least one selected from the group consisting of threonic acid, myo-inositol, and phosphorylethanolamine.

In the present specification, the term “biological sample” may be a sample isolated from a subject suffering from immune-related diseases or being at risk for immune-related diseases, or may be a cultured cell. The biological sample may be blood, plasma, serum, tissue, urine, mucus, saliva, tears, sputum, cerebrospinal fluid, pleural effusion fluid, nipple aspirate, lymph fluid, respiratory fluid, intestinal fluid, urogenital tract fluid, breast milk, lymphatic fluid, semen, cerebrospinal fluid, tracheal fluid, ascites, cystic tumor fluid, amniotic fluid, tissue, spleen, a splenocyte, spleen tissue, or a combination thereof. When the biological sample is blood or plasma, use of blood or plasma, which is easy to collect, as a specimen does not involve extraction of organ tissue of a subject, leading to easy analysis without causing a burden to a subject. The cell may be a cell isolated from a subject, or may be a cultured cell.

In an embodiment, the immunosuppression level in peripheral blood mononuclear cells or splenocytes may be measured compared to plasma, in the biological sample.

In an embodiment, the immunosuppression level may be measuring based on the level of aspartic acid metabolite.

In an embodiment, the immunosuppression level in a subject may be measured by measuring the level of aspartic acid metabolite in peripheral blood mononuclear cells or splenocytes, compared to plasma, in the biological sample.

The subject may be a mammal. The mammal may be, for example, a human, a dog, a cat, a goat, a pig, a mouse, a rabbit, a hamster, a rat, or a guinea pig.

Methods of isolating metabolites from the biological sample may be known to those skilled in the art.

In an embodiment, the biological sample may be a sample isolated from a subject suffering from immune-related disease or being at risk for immune-related diseases, or may be a cultured cell.

In an embodiment, the biological sample may be blood, plasma, a blood cell, serum, a peripheral blood mononuclear cell, a splenocyte, or a combination thereof.

In an embodiment, the immune-related diseases may include a disease involving immunosuppression or immune paralysis.

In an embodiment, the immune-related diseases may include sepsis.

In an embodiment, the method may further include determining that the biological sample exhibits immunosuppression when the level of aspartic acid metabolite is reduced compared to a control group.

In an embodiment, the aspartic acid metabolite may be selected from the group consisting of glycine, aspartic acid, L-proline, glutamic acid, and L-lysine, and may include functional equivalents of the foregoing.

In an embodiment, the determining whether the immunosuppression is exhibited may include measuring a reduction in a level of at least one selected from the group consisting of threonic acid, myo-inositol, and phosphorylethanolamine, compared to a control group.

In an embodiment, the immune-related diseases may include a disease involving immunosuppression or immune paralysis.

In an embodiment, the immune-related diseases may include sepsis.

The measuring of a level of metabolite may be performed by a method known to those skilled in the art.

To diagnose immune-related diseases and immunosuppression levels, the method may additionally utilize clinical information other than the marker for a subject, in addition to the analysis results of the marker. The clinical information may include, for example, a patient's age, gender, weight, dietary habits, and body mass index, and results of ultrasonography, computed tomography (CT), magnetic resonance imaging (MRI), and angiography, and the like.

The method may further include determining that the immunosuppression level is increased when the measured level of aspartic acid metabolite is reduced compared to a control group.

In the present specification, the term “reduced level of metabolite” may refer to a measurable and significantly reduced concentration of metabolite in a sample of a subject compared to a control group, and for example, may refer to a reduction of about 0.9-fold or less, such as a reduction of 0.1-fold to 0.9-fold, 0.9-fold, 0.8-fold, 0.7-fold, 0.6-fold, 0.5-fold, 0.4-fold, 0.3-fold, 0.2-fold, or 0.1-fold or less.

In addition, the method may further include determining that the immunosuppression level is reduced when the measured level of aspartic acid metabolite is increased compared to a control group.

In the present specification, the term “increased level of metabolite” may refer to a measurable and significantly increased concentration of metabolite in a sample of a subject compared to a control group, and for example, may refer to an increase of about 1.1-fold or more, such as 1.1-fold to 2.5-fold, 1.1-fold, 1.2-fold, 1.3-fold, 1.4-fold, 1.5-fold, 1.6-fold, 1.7-fold, 1.8-fold, 1.9-fold, or 2-fold or more.

In an embodiment, the control group may be a sample isolated from a subject or cell that has not undergone immunosuppression or immune paralysis, or a subject or cell that does not have immune-related diseases or is not at risk for immune-related diseases.

### Advantageous Effects

According to the composition and kit for diagnosing immune-related diseases in an aspect and the method of diagnosing immune-related diseases by using the composition and kit, the level of immune-related diseases in a subject may be easily diagnosed, so that the health of the subject can be monitored and the immune-related diseases can be prevented and diagnosed.

### DESCRIPTION OF DRAWINGS

FIG. 1 is an experiment design to screen for biomarkers for diagnosing an immunosuppression level.

FIG. 2 shows results of heat map analysis of levels of aspartic acid metabolites in peripheral blood mononuclear cells (PBMCs) of a control group and an intraperitoneal sepsis model, by using gas chromatography-time of flight mass spectrometry (GC-TOF-MS).

FIG. 3 is a graph showing changes in endotoxin tolerance in an intraperitoneal sepsis model, using PBMCs and splenocytes, compared to changes in plasma.

FIG. 4 is a graph measuring changes in endotoxin tolerance in an intraperitoneal sepsis model, through changes in a level of tumor necrosis factor alpha (TNF-α) in PBMCs.

FIG. 5 is a graph measuring changes in endotoxin tolerance in an intraperitoneal sepsis model, through changes in a level of TNF-α in splenocytes.

FIG. 6 is a graph showing endotoxin tolerance in PBMCs of each rat group according to the severity of sepsis.

FIG. 7 is a graph showing endotoxin tolerance in splenocytes of each rat group according to the severity of sepsis.

### MODE FOR INVENTION

Hereinafter, the present disclosure will be described in more detail with reference to Examples below. However, these Examples are for illustrative purposes of one or more embodiments, and the scope of the present disclosure is not limited thereto.

### EXPERIMENTAL EXAMPLE 1

### Screening for Immunosuppression-Related Biomarkers

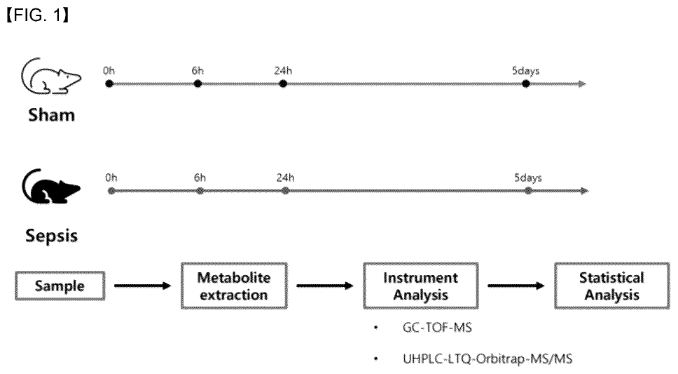

An experiment design to screen for immunosuppression-related biomarkers by using peripheral blood mononuclear cells (PBMCs) and splenocytes is shown in FIG. 1. By measuring contents of metabolites before and after induction of immunosuppressive responses, immunosuppression-related biomarkers were derived. Targeting an intraperitoneal sepsis model and a rat of a control group, contents of metabolites in PBMCs and splenocytes were analyzed, and afterwards, through public databases using the analyzed metabolites used as raw data, a group of candidates for the immunosuppression-related biomarkers was derived.

FIG. 1 is an experiment design to screen for biomarkers for diagnosing an immunosuppression level.

### 1.1. Experimental Animals and Treatment

Male Sprague-Dawley rats weighing 270 to 300 grams (g) were used to create cecal slurry models, which are intraperitoneal sepsis-bearing models known worldwide. Sepsis was induced by obtaining feces from a feces donor rat, and then dividing the feces into a certain amount and injecting the feces into a sepsis-bearing rat by opening the abdominal cavity thereof. Specifically, the feces donor rat was anesthetized with intramuscular injections of Zoletil (50 mg/kg) and xylazine (10 mg/kg), and then was subjected to a midline laparotomy to extrude the cecum. A 0.5 cm incision was made on the anti-intestinal membrane surface of the cecum, and the cecum was squeezed to excrete feces. The feces were collected, weighed, and diluted with a 5% dextrose saline solution at a ratio of 1:3. A sepsis-bearing rat was anesthetized by using the aforementioned method, and a 10.5 cm midline laparotomy was performed thereon. Then, the fecal slurry was administered intraperitoneally. Before the intraperitoneal administration, the fecal slurry was vortexed to obtain a homogeneous suspension. The volume of fecal slurry to be administered to each animal was adjusted based on the body weight of the sepsis-bearing rat. After sepsis induction, treatment for sepsis included subcutaneous fluid resuscitation (30 ml/kg of 5% dextrose saline solution) and subcutaneous injection of antibiotics, such as imipenem, at a dose of 25 mg/kg twice daily for 2 days.

Blood and spleen were collected from rats for analysis of metabolites at 6 hours and 24 hours and on Day 5 after the trigger of sepsis. PBMCs were obtained from the collected blood, whereas splenocytes were obtained from the spleen. In a control group, analysis of metabolites was performed on the same sample obtained at 6 hours and 24 hours and on Day 5 from rats not triggering sepsis.

### 1.2. Analysis of Metabolites in Samples

To analyze metabolites in the PBMC sample and the splenocyte sample obtained in Experimental Example 1.1, analysis was performed by using gas chromatography-time of flight mass spectrometry (GC-TOF-MS) as follows.

Specifically, the metabolite sample obtained in Example 1.1 was mixed with 1,000 uL of methanol (JTBaker, 9093-68) to which 10 uL of 2-chloro-l-phenylalanine (SigmaAldrich, 47766), an internal standard material, was added, and then homogenized for 10 minutes by using the Mixermill (SPEX SamplePrep, 8000M). After the homogenization, the supernatant was sonicated for 1 minute and stored at 4° C. for 1 hour. The supernatant was then centrifuged at 13,500 xg at 4° C. for 10 minutes and filtered through 0.2 μm polytetrafluoroethylene (PTFE) (Sigma-Aldrich, P0325). The filtrate was dried and treated with 50 uL of methoxyamine hydrochloride (Sigma-Aldrich, 226904) for 30 to 90 minutes to prepare oximes. 50 uL of N-methyl-N-trimethylsilyl-trifluoroacetamide (Sigma-Aldrich, 69482) was added thereto for derivatization and allowed for a reaction at 37° C. for 30 minutes, and the reaction product was analyzed by GC-TOF-MS.

The GC-TOF-MS analysis was performed by using the Agilent 7890 gas chromatograph system (Agilent Technologies, Palo Alto, CA, USA) and the Rtx-5MS column (i.d., 30 m×0.25 mm, 0.25 μm particle size; Restek Corp., Bellefonte, PA, USA). According to the protocol provided by the manufacturer, helium was constantly flowed at 1.5 mL/min and 1 μL of the reactant sample was injected. Here, the temperatures of the front inlet and transfer line were maintained at 250° C. and 240° C., respectively, electron ionization was performed at −70 eV, and data were collected in the range of 50 to 1000 m/z.

### 1.3. Statistical Analysis

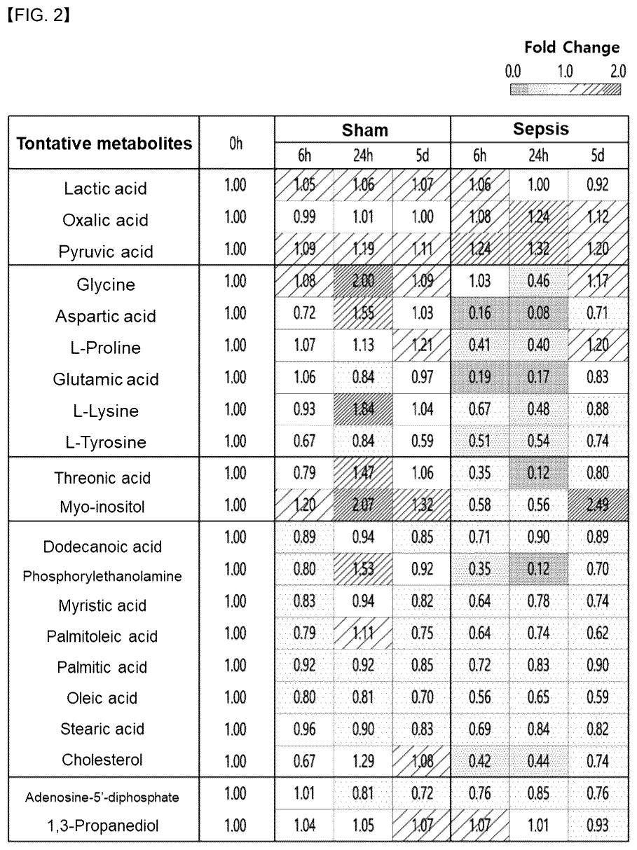

To interpret the GC-TOP-MS analysis results of Experimental Example 1.2, hierarchy analysis was performed to create a heat map, and results thereof are shown in FIG. 2. The difference in the contents of metabolites in the two groups was expressed as a fold of the contents of metabolites in the experimental group and the control group, with time 0 as the baseline (1.00).

FIG. 2 shows results of heat map analysis of levels of aspartic acid metabolites in PBMCs of a control group and an intraperitoneal sepsis model, by using GC-TOF-MS.

As shown in FIG. 2, the relative content of aspartic acid metabolites was confirmed to be higher in the control group where sepsis was not induced, compared to the experimental group which was an intraperitoneal sepsis model.

### 1.4. Selection of Aspartic Acid Metabolites

As shown in FIG. 2, the hierarchy analysis was performed on metabolites including amino acids, organic acids, fatty acids, and the like, and eight aspartic acid metabolites showing statistically significant differences in the experimental group compared to the control group were finally selected through the heat map.

The eight selected aspartic acid metabolites are glycine, aspartic acid, L-proline, glutamic acid, L-lysine, threonic acid, myo-inositol, and phosphorylethanolamine, and the levels of the eight metabolites were significantly reduced in the sepsis-bearing model compared to the control group in the early stage of sepsis.

In addition, it was observed that the contents of aspartic acid metabolites that have been decreased after sepsis induction showed the largest difference at 24 hours after sepsis induction and that the difference was recovered thereafter.

Referring to the results above, it was confirmed that the eight selected aspartic acid metabolites are biomarkers available for diagnosing sepsis or immune-related diseases involving immunosuppression.

### EXPERIMENTAL EXAMPLE 2

### Verification of Endotoxin Tolerance in Diagnosis of Immunosuppression Levels

Endotoxin tolerance refers to decreased responsiveness to LPS after initial exposure to endotoxins. This has been proposed as one of the underlying mechanisms causing immunosuppression or immune paralysis in sepsis. (Rearte B et al., Differential effects of glucocorticoids in the establishment and maintenance of endotoxin tolerance., 2010).

To verify immunosuppression occurring in the intraperitoneal sepsis model through endotoxin tolerance, the concentration of TNF-α after LPS stimulation in PBMCs and splenocytes was measured at 6 hour, 12 hour, 24 hour, 48 hour, and 72 hours and on Day 5 day and Day 7 after induction of intraperitoneal sepsis.

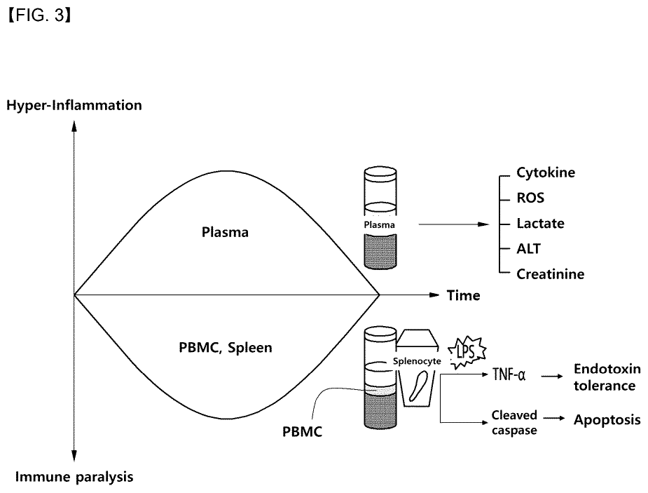

FIG. 3 is a graph showing changes in endotoxin tolerance in an intraperitoneal sepsis model, using PBMCs and splenocytes, compared to changes in plasma.

2.1. Measurement of Changes in Endotoxin Tolerance in PBMCs after Sepsis Induction

PBMCs were isolated from the blood of a control group (n=9) and rats sacrificed at 6 hours (n=11), 12 hours (n=11), 24 hours (n=12), 48 hours (n=5), and 72 hours (n=6) and on Day 5 (n=6) and Day 7 (n=6) after sepsis induction. PBMCs were isolated by a centrifugation method (e.g., ficoll gradient). The isolated PBMCs were stimulated with LPS to observe and compare the immunosuppression levels. Levels of TNF-α were measured 5 hours after LPS stimulation. The isolated PBMCs were seeded at a density of 1×10⁵ cells/mL onto a 96-well plate, and 100 ng/ml of LPS (Escherichia coli O111:B4, Sigma-Aldrich, St. Louis, MO, USA) was added to each well. After 5 hours, the culture medium was obtained, and changes in the levels of TNF-α were analyzed by using the TNF-α ELISA kit (ab236712, Cambridge, Abcam, MA, USA).

FIG. 4 is a graph measuring changes in endotoxin tolerance in an intraperitoneal sepsis model, through changes in the level of TNF-α levels in PBMCs.

As shown in FIG. 4, after LPS stimulation in PBMCs, the TNF-α response was significantly decreased until 48 hours after sepsis, and then gradually recovered thereafter, and completely recovered on Day 5 after sepsis.

### 2.2. Measurement of Changes in Endotoxin Tolerance in Splenocytes After Induction of Sepsis

Splenocytes were isolated from the blood of a control group (n=11) and rats sacrificed at 6 hours (n=18), 12 hours (n=12), 24 hours (n=29), 48 hours (n=11), and 72 hours (n=16) and on Day 5 (n=6) and Day 7 (n=6) after sepsis induction. The isolated splenocytes were stimulated with LPS to observe and compare the immunosuppression levels. Levels of TNF-α were measured 5 hours after LPS stimulation. The isolated splenocytes were seeded at a density of 1×10⁵ cells/mL onto a 6-well plate, and 11 μg/mL of LPS (Escherichia coli O111:B4, Sigma-Aldrich, St. Louis, MO, USA) was added to each well. After 5 hours, the culture medium was obtained, and the levels of TNF-α were analyzed by using the TNF-α ELISA kit (R&D Systems, Inc., Minneapolis, MN, USA).

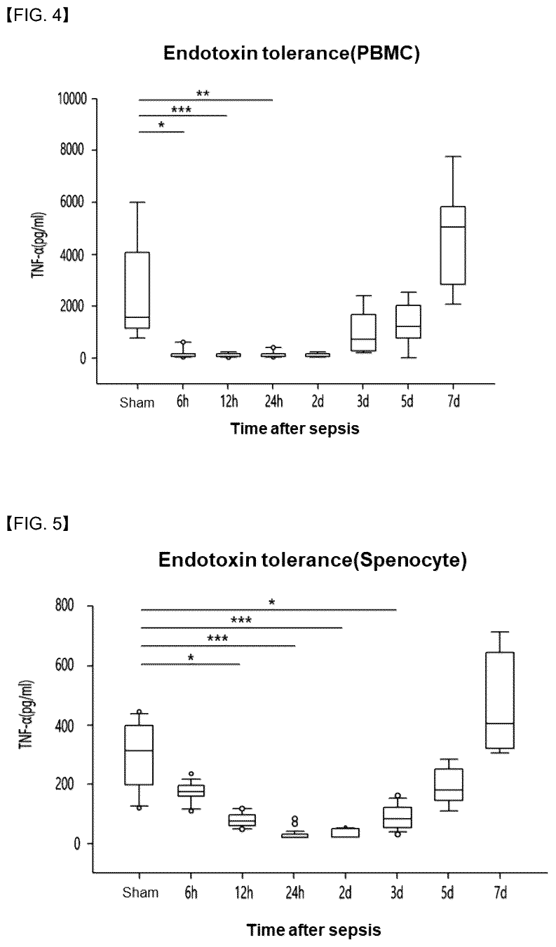

FIG. 5 is a graph measuring changes in endotoxin tolerance in an intraperitoneal sepsis model, through changes in the levels of TNF-α in splenocytes.

As shown in FIG. 5, after LPS stimulation in splenocytes, the release of TNF-α was also significantly decreased until 24 hours after sepsis, and tended to gradually recover thereafter, and then completely recovered on Day 7 after sepsis.

### 2.3. Endotoxin Tolerance According to Severity of Induced Sepsis

To determine the effect of sepsis severity on endotoxin tolerance, mild and severe sepsis models were induced by using a fecal slurry at different volumes, and 24 hours after induction of sepsis, endotoxin tolerance was measured in PBMCs and splenocytes, respectively.

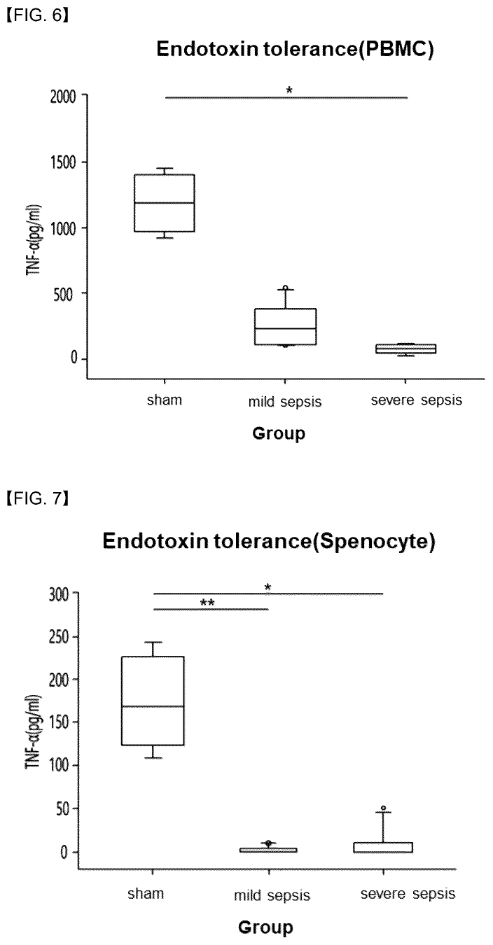

FIG. 6 is a graph showing endotoxin tolerance in PBMCs of each rat group according to the severity of sepsis.

FIG. 7 is a graph showing endotoxin tolerance in splenocytes of each rat group according to the severity of sepsis.

As shown in FIGS. 6 and 7, endotoxin tolerance in PBMCs and splenocytes was observed more prominently in the model induced with severe sepsis. Here, the mortality rates in the mild sepsis model and the severe sepsis model were 0% and 50%, respectively.

Referring to the results above, endotoxin tolerance was observed starting 6 hours after induction of sepsis, and afterwards, reached the lowest point between 24 hours and 48 hours, and then tended to recover.

In summary, these results confirm that the contents of the eight selected aspartic acid metabolites tend to recover after a decrease in endotoxin tolerance, which is known to be an underlying mechanism of immunosuppression, in the sepsis-bearing model, and likewise, the contents of the eight aspartic acid metabolites were confirmed to recover after a decrease in the sepsis-bearing model. Accordingly, it is suggested that the eight selected aspartic acid metabolites can be used as biomarkers for diagnosing immunosuppression and diseases involving immunosuppression such as sepsis.

1.-5. (canceled)

6. A method of diagnosing an immunosuppression level in a subject, the method comprising measuring a level of aspartic acid metabolite in a biological sample separated from a subject.

7. The method of claim 6, wherein the aspartic acid metabolite is at least one metabolite selected from the group consisting of glycine, aspartic acid, L-proline, glutamic acid, and L-lysine.

8. The method of claim 6, wherein the diagnosing an immunosuppression level comprises measuring a level of at least one selected from the group consisting of threonic acid, myo-inositol, and phosphorylethanolamine.

9. The method of claim 6, wherein the biological sample is a sample isolated from a subject suffering from immune-related diseases or being at risk for immune-related diseases, or is a cultured cell.

10. The method of claim 6, wherein the biological sample is blood, plasma, a blood cell, serum, a peripheral blood mononuclear cell, a splenocyte, or a combination thereof.

11. The method of claim 9, wherein the immune-related diseases include sepsis.

12. The method of claim 6, further comprising determining that the biological sample exhibits immunosuppression when the level of aspartic acid metabolite is reduced compared to a control group.

13. The method of claim 10, wherein the immunosuppression level in a peripheral blood mononuclear cell or a splenocyte is measured, compared to plasma, in the biological sample.

14. The method of claim 13, wherein the immunosuppression level is measured by measuring the level of aspartic acid metabolite.
